(19) United States
(12) Patent Application Publication
(10) Pub. No.: US 2017/0347993 A1
(43) Pub. Date: Dec. 7, 2017

(54) SYSTEM AND METHOD FOR ULTRASOUND CUSTOMIZATION

(71) Applicant: CARESTREAM HEALTH, INC., Rochester, NY (US)

(72) Inventor: Ajay ANAND, Rochester, NY (US)

(21) Appl. No.: 15/299,878

(22) Filed: Oct. 21, 2016

Related U.S. Application Data
(60) Provisional application No. 62/346,027, filed on Jun. 6, 2016.

(51) Int. Cl. A61B 8/00; A61B 34/00; G06N 99/00

(52) U.S. Cl. CPC A61B 8/585; G06N 99/005; A61B 8/467; A61B 34/25; A61B 8/465; A61B 2034/258

(57) ABSTRACT

A method tracks a user's operation of an ultrasound system. Based on the tracked operations, the method uses a machine-learning module to generate a proposed custom system configuration for the ultrasound system for the user. The proposed custom system configuration is presented to the user. In response to a user instruction, the proposed custom system configuration is implemented on the ultrasound system.

### CROSS REFERENCE TO RELATED APPLICATIONS

This application claims priority to U.S. Provisional application U.S. Ser. No. 62/346,027, provisionally filed on Jun. 6, 2016 entitled “SYSTEM AND METHOD FOR ULTRASOUND CUSTOMIZATION”, in the name of Anand, incorporated herein in its entirety.

### FIELD OF THE INVENTION

The invention relates generally to medical ultrasound systems and methods, and in particular to a method for workflow for ultrasound apparatus operation.

### BACKGROUND

Ultrasound imaging systems/methods are known, such as those described, for example, in U.S. Pat. No. 6,705,995 (Poland), U.S. Pat. No. 5,370,120 (Oppelt), and U.S. Pat. No. 8,285,357 (Gardner), all of which are incorporated herein in their entirety. Various applications for diagnostic ultrasound systems are given, for example, in the article entitled “Ultrasound Transducer Selection In Clinical Imaging Practice”, by Szabo and Lewin, Journal of Ultrasound Medicine, 2013; 32:573-582, incorporated herein by reference in its entirety.

Ultrasound utilizes sound waves at frequencies higher than those perceptible to the human ear. Ultrasonic images known as sonograms are generated as a result of pulsed ultrasonic energy that has been directed into tissue using a probe. The probe obtains echoed sound energy from the internal tissue and provides signal content that represents the different sound reflectivity exhibited by different tissue types. This signal content is then used to form images that visualize features of the internal tissue. Medical ultrasound, also known as diagnostic sonography or ultrasonography, is used as a diagnostic imaging technique used to help visualize features and operation of tendons, muscles, joints, vessels and internal organs of a patient.

FIGS. 1A-1B and FIGS. 2-3 show exemplary portable ultrasound systems 10 having a cart/base/support 12, a display or display/monitor 14, one or more input interface devices 16 (such as keyboard or mouse), and a generator 18. The display/monitor 14 can be a touchscreen in order to function as an input device. As illustrated, the ultrasound system 10 can be a mobile or portable system designed to be wheeled from one location to another. As illustrated in FIG. 2, ultrasound system 10 has a central processing unit CPU 20 that provides control signals and processing capabilities. CPU 20 is in signal communication with display 14 and interface device 16, as well as with a storage device 22 and an optional printer 24. A transducer probe 26 provides the ultrasound acoustic signal and generates an electronic feedback signal indicative of tissue characteristics according to the echoed sound.

FIG. 3 shows an example of an ultrasound system 10 in use with an image displayed on display 14.

Different types of images, with different appearance, can be formed using sonographic apparatus. Monochrome B-mode image displays the acoustic impedance of a two-dimensional cross-section of tissue. Other types of image can use color or other types of highlighting to display specialized information such as blood flow, motion of tissue over time, the location of blood, the presence of specific molecules, tissue stiffness, or the anatomy of a three-dimensional region.

Accordingly, the ultrasound systems of FIGS. 1A-3 are typically configured to operate within at least two different ultrasound modes. As such, the system provides means to switch between the at least two different ultrasound modes. Such a multi-mode configuration, along with techniques for switching between modes, are known to those skilled in ultrasound technology.

In conventional workflow, the sonographer or other operating practitioner begins an examination with B-mode imaging in order to locate the anatomy or region of interest (ROI). B-mode imaging is relatively unconstrained, providing at least sufficient information for identifying prominent anatomical features. Then, once the ROI is located, the sonographer switches to a suitable imaging mode for the particular requirements of an exam, in which more specialized signals and signal sensing may be used. In switching from one mode to the next, however, the sonographer must often readjust various equipment settings and may need to manually identify or adjust the ROI for the new mode. For example, there can be portions of the ROI that either require special imaging treatment or that simply can't be acceptably imaged using a particular mode. The need for this type of tedious and repeated adjustment complicates sonographer workflow, adding time and steps to the procedure to obtain the desired image.

Sonography apparatus are designed to provide numerous functions and utilities to the operator. While these systems offer significant capability and flexibility, however, the operator is often required to enter repeated sequences of instructions in order to perform standard functions. In practice, the operator typically needs to enter instructions with one hand while holding the transducer probe in the other, as shown in FIG. 3. Different operators may develop and use different sequences of operations, using workflows that allow them to work more comfortably and efficiently for particular equipment and surroundings. Operators, moreover, can have different styles for working with the ultrasound system and may find it more convenient to be on one side of the control console or the other depending on the task or on patient position, or based on whether the operator is right-handed or left-handed or prefers to work from one side of the patient or the other. Positioning and other practical factors can dictate how the operator works most comfortably and efficiently with the ultrasound system for various types of exams.

Accordingly, there is a desire to provide ways to improve workability and workflow for the sonographer and to address the need for workflow customization that can help to improve operator comfort and efficiency.

### SUMMARY

According to one aspect of the invention, there is provided a system and method for ultrasound imaging. An object of the present disclosure is to advance the art of ultrasound imaging and to provide a method and apparatus for customization of the operator interface that is better adapted to the workflow and practices of individual sonographers at a particular imaging site.

These aspects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to an embodiment of the present disclosure, there is provided a method comprising: tracking a user's operation of an ultrasound system; based on the tracked operations, using a machine-learning module to generate a proposed custom system configuration for the ultrasound system for the user; presenting the proposed custom system configuration to the user; and in response to a user instruction, implementing the proposed custom system configuration on the ultrasound system.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A and 1B show exemplary ultrasound systems.

FIG. 2 shows a schematic of an exemplary ultrasound system.

FIG. 3 illustrates a sonographer using an exemplary ultrasound system.

FIG. 4 shows a displayed ultrasound image having a default region of interest, shown in grayscale.

FIG. 5 shows a displayed ultrasound image having a region of interest shown as a bounded rectangle, wherein features within the region of interest are highlighted in color.

FIG. 6 is a schematic diagram that shows functional structures of the CPU that provides control of the operator configuration for an ultrasound system.

FIG. 7 shows a diagram of a sign-on screen for the sonography operator.

FIG. 8 shows a diagram of a security authorization screen displayed upon login to the system.

FIGS. 9A and 9B are two parts of a logic flow diagram showing a sequence for customization of ultrasound system operation.

FIG. 10 shows an exemplary display screen arrangement for viewing and accepting user interface changes for operator customization.

### DETAILED DESCRIPTION OF EXEMPLARY EMBODIMENTS

The following is a detailed description of the embodiments of the invention, reference being made to the drawings in which the same reference numerals identify the same elements of structure in each of the several figures.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may convey physical, electrical, magnetic, electromagnetic, optical, wired, and/or wireless signals between the first device and/or component and second device and/or component. The signal paths may also include additional devices and/or components between the first device and/or component and second device and/or component.

In the context of the present disclosure, the term “subject” or “body” or “anatomy” is used to describe a portion of the patient that is undergoing ultrasound imaging. The terms “sonographer”, “technician”, “viewer”, “user”, “operator”, and “practitioner” are used equivalently to indicate the person who actively operates the sonography equipment.

The term “highlighting” for a displayed element or feature has its conventional meaning as is understood to those skilled in the information and image display arts. In general, highlighting uses some form of localized display enhancement to attract the attention of the viewer. Highlighting a portion of a display, such as a particular value, graph, message, or other element can be achieved in any of a number of ways, including, but not limited to, annotating, displaying a nearby or overlaying symbol, outlining or tracing, display in a different color or at a markedly different intensity or grayscale value than other image or information content, blinking or animation of a portion of a display, or display at larger scale, higher sharpness, or contrast.

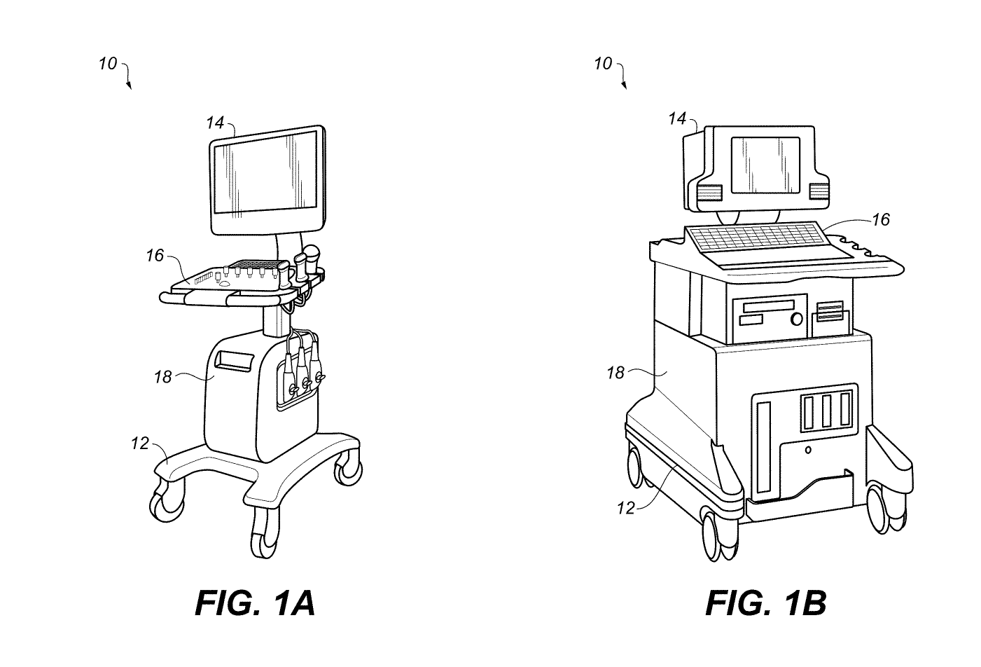

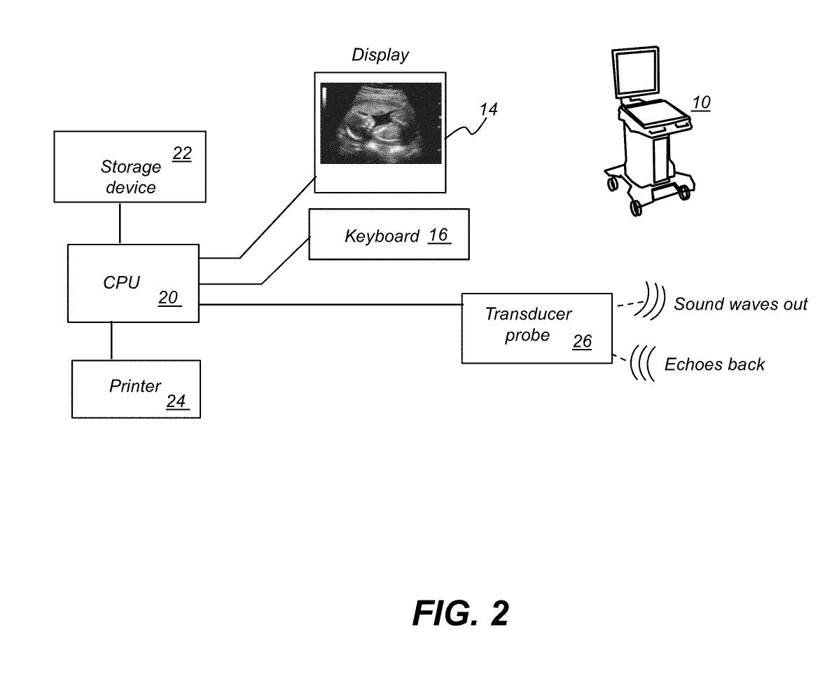

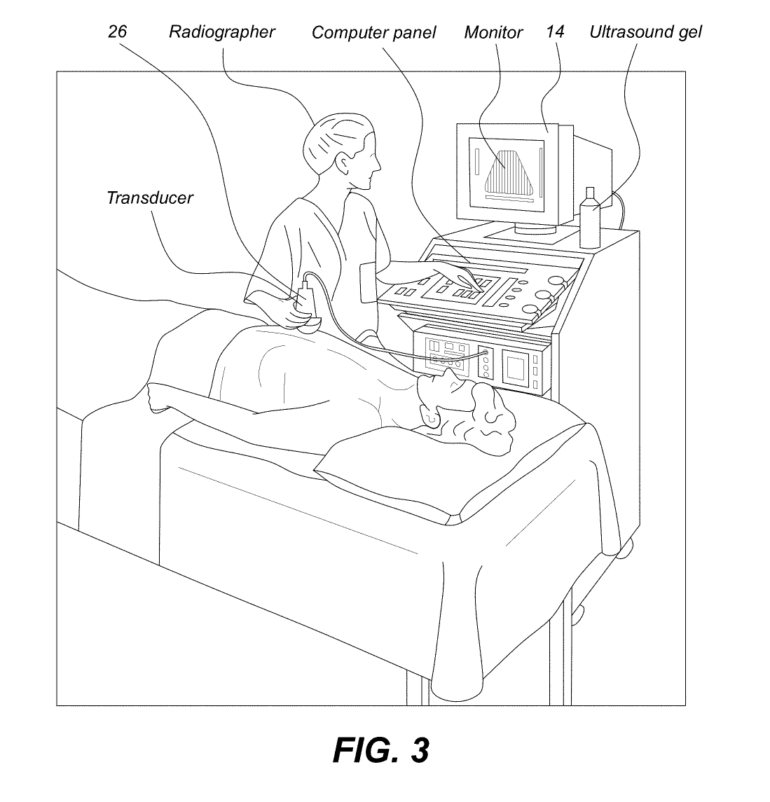

The ultrasound system, shown by way of example in FIGS. 1A and 1B, can include image processing system, a user interface and a display. The image processing system includes a memory and a processor. Additional, different, or fewer components may be provided in the overall system or in the image processing chain. In one embodiment, the system is a medical diagnostic ultrasound imaging system. The memory is a RAM, ROM, hard drive, removable media, compact disc, DVD, floppy disc, tape, cache memory, buffer, capacitor, combinations thereof or any other now known or later developed analog or digital device for storing information. The memory is operable to store data identifying a selected point for identifying a region of interest. The memory is operable to store data identifying one or a plurality of region of interest. Information from the user interface indicating a position on an image on the display is used to determine a spatial relationship of a user selected point to a scanned region or image position. The selected point is an individual or single point in one embodiment that may be a point selected within a line, area or volume. Additional or different information may be also stored within the memory.

The processor is general processor, application specific integrated circuit, digital signal processor, controller, field programmable gate array, digital device, analog device, transistors, combinations thereof or other now known or later developed devices for receiving analog or digital data and outputting altered or calculated data. The user input is a track ball, mouse, joy stick, touch pad, buttons, slider, knobs, position sensor, combinations thereof or other now known or later developed input devices. The user input is operable to receive a selected point from a user. For example, the user positions a cursor on an image displayed on the display. The user then selects a position of the cursor as indicating a point for a region of interest. The display is a CRT (cathode-ray tube), LCD (liquid crystal device), plasma screen, projector, combinations thereof, or other now known or later developed devices for displaying an image, a region of interest, region of interest information and/or user input information. The display can be a touch screen display that includes a keyboard having a programmable layout.

Modes of ultrasound used in medical imaging include the following:
A-mode: A-mode (amplitude mode) is the simplest type of ultrasound. A single transducer scans a line through the body with the echoes plotted on screen as a function of depth. Therapeutic ultrasound aimed at a specific tumor or calculus is also A-mode, to allow for pinpoint accurate focus of the destructive wave energy.
B-mode or 2D mode: In B-mode (brightness mode) ultrasound, a linear array of transducers simultaneously scans a plane through the body that can be viewed as a two-dimensional image on screen. Sometimes referred to as 2D mode, this mode is generally the starting point for exam types that use other modes.
C-mode: A C-mode image is formed in a plane normal to a B-mode image. A gate that selects data from a specific depth from an A-mode line is used; then the transducer is moved in the 2D plane to sample the entire region at this fixed depth. When the transducer traverses the area in a spiral, an area of 100 cm² can be scanned in around 10 seconds.
M-mode: In M-mode (motion mode) ultrasound, a sequence of pulses is emitted, with pulses in quick succession. With each pulse, either an A-mode or B-mode image is acquired. Over time, M-mode imaging is analogous to recording a video in ultrasound. As the organ boundaries that produce reflections move relative to the probe, this mode can be used to determine the velocity of specific organ structures.
Doppler mode: This mode makes use of the Doppler effect in measuring and visualizing blood flow.
Color Doppler: Velocity information is presented as a color-coded overlay on top of a B-mode image. This mode is sometimes referred to as Color Flow or color mode.
Continuous Doppler: Doppler information is sampled along a line through the body, and all velocities detected at each point in time are presented (on a time line).
Pulsed wave (PW) Doppler: Doppler information is sampled from only a small sample volume (defined in 2D image), and presented on a timeline.
Duplex: a common name for the simultaneous presentation of 2D and (usually) PW Doppler information. (Using modern ultrasound machines, color Doppler is almost always also used; hence the alternative name Triplex.).
Pulse inversion mode: In this mode, two successive pulses with opposite sign are emitted and then subtracted from each other. This implies that any linearly responding constituent will disappear while gases with non-linear compressibility stand out. Pulse inversion may also be used in a similar manner as in Harmonic mode.
Harmonic mode: In this mode a deep penetrating fundamental frequency is emitted into the body and a harmonic overtone is detected. With this method, noise and artifacts due to reverberation and aberration are greatly reduced. Some also believe that penetration depth can be gained with improved lateral resolution; however, this is not well documented.
Elastography mode: this mode maps the elastic properties of soft tissue. The main idea is that whether the tissue is hard or soft will give diagnostic information about the presence or status of disease. For example, cancerous tumors will often be harder than the surrounding tissue, and diseased liver tissues are found to be stiffer than healthy ones.

While conducting an ultrasound exam, the sonographer may often switch between multiple ultrasound modes. In conventional practice, for example, the sonographer first operates in a B-mode in order to coarsely locate the ROI. The sonographer then transitions to a Doppler mode before moving back to the B-mode. For some particular examinations, there are pre-set (or pre-determined or pre-defined) steps and a predetermined sequence of modes that the sonographer must follow. That is, the ordered interaction sequence of modes and setup instructions used in a particular exam type can be predefined for the operator as part of an operator protocol for the exam.

For carotid artery imaging, for example, the exam typically follows a progression of modes such as the following:

(i) B-mode for initial positioning and establishing reference coordinates of the sample volume;

(ii) Color Flow mode for improved visualization of blood vessels; and

(iii) Pulse wave Doppler mode for highlighting blood flow within the sample volume.

For heart imaging, for example, the exam protocol can use a progression that begins with B-mode or M-mode imaging for auto-positioning of the cursor, followed by Color Flow or pulse wave Doppler modes.

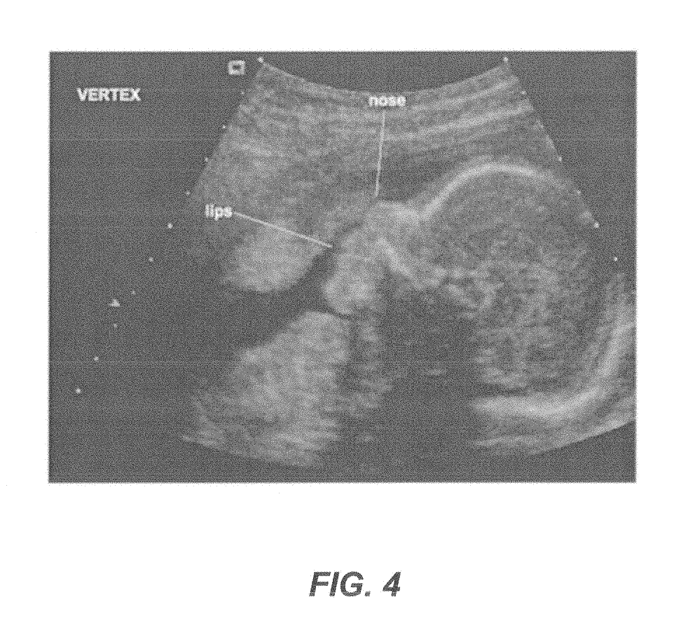

The sonography workflow typically begins with acquisition and display of a grayscale mode image (such as the B-mode image illustrated in FIG. 4) in order to survey the anatomy. FIG. 4 shows an exemplary B-mode ultrasound image, displayed as a grayscale image.

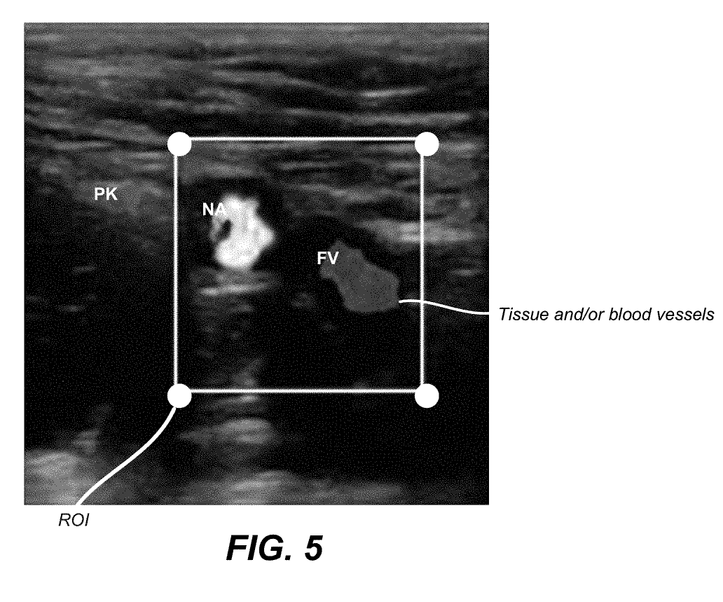

FIG. 5 shows an image with an ROI (region of interest) that is outlined and has color highlighting for tissue and blood vessels, obtained in Color Flow mode.

Within the ultrasound image on the display, the particular area of the displayed image that is of interest to the sonographer or other practitioner is referred to as the Region of Interest (ROI) or, alternately, the ROI extent. As the sonographer conducts the examination and switches between imaging modes, the displayed size and position, as well as the apparent shape of the ROI may change.

The region of interest (ROI) can be defined in any of a number of ways. In conventional practice, as shown in the example of FIG. 5, the ROI is defined by multiple points or vertices that define a polygon shape, such as defining a rectangle or other parallelogram by its four corners, for example. Alternately, the ROI can be defined by a point and a distance, such as a center point and a radius or function of the distance from the point to a single boundary. The distance may be, for example, any of a radius, circumference, diagonal, length or width, diameter or other characteristic of a shape. The region of interest can alternately be defined by a point and two distances, such as a distance to each of two boundaries. In another arrangement, the region of interest can be a pre-defined shape positioned around a point, such as a square, rectangle, oval or combination thereof.

An embodiment of the present disclosure addresses the need for control of accessibility to various functions for the ultrasound system operator as well as for maintaining operator information for users of the ultrasound system and generating and maintaining operator profiles customized according to detected use patterns. To support these and related functions, the ultrasound system 10 according to an embodiment of the present invention provides mechanisms for identifying at least levels of function permitted to a user and for providing controlled customization of the user interface for the system.

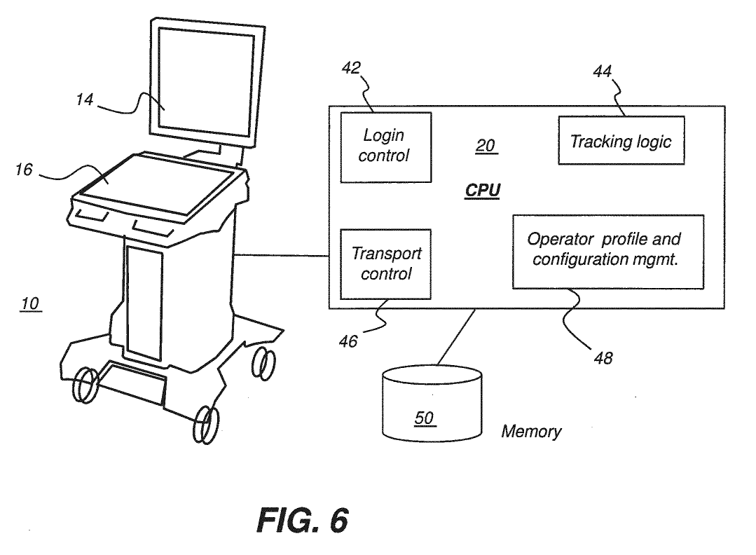

Referring to FIG. 6, there is shown, in schematic form, some of the functional structures of CPU 20 that can optionally provide this added measure of control and configuration flexibility. Login control logic 42 comprises hardware and software components that provide operator identification and validation and that can maintain a correlation between operator identification and access permission to system features, as described in more detail subsequently. Transport control logic 46 allows control of system transport according to operator permissions. This can include the capability to lock or unlock system brakes or assisting motor for system transport, where provided. Tracking logic 44 provides functions for monitoring and recording operator activity with the system, such as control settings and entry of a sequence of instructions for a particular task. Operator profile and configuration logic 48 manages personal profile information for each authorized operator and has the capability to generate and adapt system configuration according to the operator profile and operator activity, as reported by tracking logic 44. A memory 50 provides short-term and long-term storage to support the operational management features described herein for CPU 20.

### Login and Operator Identification

As described previously, ultrasound system 10 provides an operator login function that identifies the operator and that enables the identified operator to have appropriate capabilities for deploying and using the system. According to an embodiment of the present invention, there can be different authorization levels, set up for the operators at a site by an administrative user. A first class of operators is permitted to unlock and move the portable system 10 to another location; however, operation, or operation in certain modes, is not permitted to this class of operators. A second class of operators also has the unlocking and movement permissions of the first class, adding the capability for full deployment and operation of the system.

According to an alternate embodiment of the present disclosure, a single sign-on capability allows any user registered to the system to move or operate the system without restriction.

More complex permissions arrangements are possible. The system administrator can set up individual system features accessible to different operators depending on skill level or on a need-to-know basis. Some personnel, for example, are only permitted to move the ultrasound system 10 from place to place. Others have only the capability to view stored images. Another group has full capability for transport, operation, viewing, and storage or uploading of acquired images. Alternately, the ultrasound system can have multiple logins, one for system transport, another for viewing or operation.

Ultrasound systems can be modified to use a single secure login to the apparatus to control system access from any of a number of aspects, for example:

(i) a transport mechanism can be enabled or disabled according to operator login;

(ii) the ultrasound generator or beam-former can be enabled or disabled based on login;

(iii) remote control and image processing control can be enabled or disabled according to operator login; or

(iv) secured storage can be provided for transducers or other needed components, locked within the system or locked out of system connection except where login permits.

Embodiments according to this disclosure can be directed to a single sign-on or logon capability for ultrasound systems, particularly such systems which are portable or mobile and that may be shared among a number of users. Exemplary single sign-on embodiments can combine turning on (e.g., power on, key start) of the portable system or a computer login (e.g., main console or display) where the computer logon action can complete a transport enablement procedure, allow generator/source control operations access and use, and/or control a lockable storage compartment for probes and other components.

In one embodiment, a single sign-on can be a single keyboard/keypad logon by an operator to activate an ultrasound system and a user interface by employing a single action. Once the user/operator has logged on (e.g., signed in) to the system, the operator will be able to drive or otherwise move the system, view images, acquire images, and perform all other operations provided or allowable to that operator.

Exemplary single sign-on mechanisms can include, but are not limited to, keyboard or keypad entry of a password or personal ID number (PIN) logon; entry of a user name plus password logon as used with conventional computer devices; entry from a card reader scan such as from a smart card, a magnetic stripe card, bar code data, or from a proximity reader that can be compatible with access technologies such as RFID, Bluetooth, or other wireless communication device; entry using a proximity card; sensing of a wireless smart card; detection of a Wiegand format card; entry from a magnetic reader device or card; a scan from an optical reader device or card; data from an infrared reader device or card; or biometric data such as a fingerprint, a retinal scan or the like.

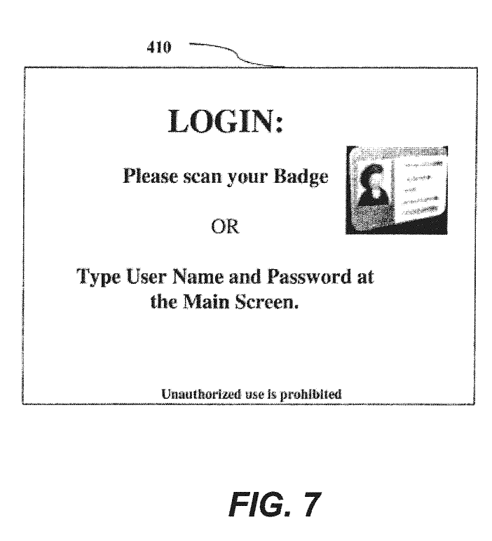

FIG. 7 illustrates an embodiment of a single sign-on screen according to an embodiment of the present disclosure. When an attempt is made to operate the ultrasound system, a single sign-on screen 410 can be displayed to provide login instructions to a user. As shown in FIG. 7, the single sign-on screen 410 can provide instructions for single sign-on such as the login message shown in FIG. 7. Preferably, single sign-on passwords can expire periodically, such as monthly, and can thereafter be renewed regularly for additional security. In addition, an unauthorized use warning can be displayed.

Alternate sign-on methods can be used. For example, the sonographer ID badge can be scanned to perform the single logon to the system. Scanning can be initiated by placing the ID badge in close proximity to a login reader that is in signal communication with the ultrasound system.

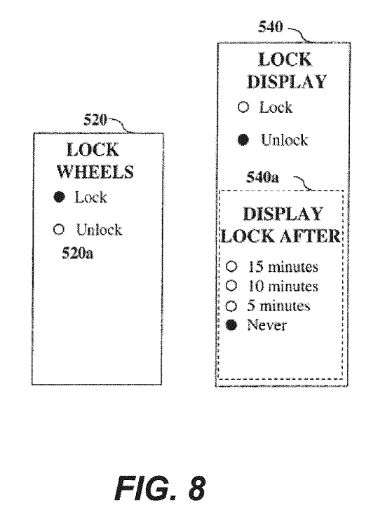

Referring to FIG. 8, there is shown, in one embodiment, a response screen that follows operator login using a sign-on screen. Verified identification using the logon can provide authorization to access the first display, access the lockable storage, and access the transport drive mechanism using wheels (e.g., a mechanism on the handle can be enabled). Further, security features for control of single logon authorizations can be managed using an embodiment of a security authorization screen that can be accessed from a main screen or window on the display.

As shown in FIG. 8, a security authorization screen that displays following login can include a command entry option for a lockable storage section, a lockable transport selection 520, and a lockable console or display selection 540. The lockable transport selection 520 can provide the authorized operator, once logged in, with the capability of locking the mobile system. A section 540a can provide options for locking the display screen.

According to an exemplary embodiment, the single sign-on capability can allow a sonographer, custodian, or medical staff member to scan an ID badge for sign-on authorization and positive identify verification. When authorized, the system can then allow manual transport of the unit. In another embodiment, a verified sign-on can be obtained using a keypad entry at a system display. With entry of the proper access code, the operator is enabled to unlock the transport mechanism (e.g., wheels) only. Such a limited functionality provided by a verified sign-on by the single sign on the reader can allow for secured, but limited, access to the mobile system in a medical facility when needed (e.g., an emergency or a fire). Further, a lockable console selection can provide a secured sleep mode whereby the display is locked after a set time interval (e.g., variable) of inactivity.

Conventional administrative and support utilities can be provided for control of operator authorization, using methods known to those skilled in the information technology arts. For example, an administrative user can have a separate logon that allows the administrator to set up an account for each operator and to store authorization information as part of the account data. An operator profile can be generated by the system in order to store authorization information for each operator who has been registered to the system by the administrator.

### Customization for the Logged In Operator

By maintaining an operator profile for each registered operator, with a custom system configuration, system customization can be expanded beyond transport control to encompass other operational aspects of the system. An ultrasound system can have a large number of user parameters for image acquisition that are often manually configured by each user during the interaction sequence or that may be set as defaults at a departmental level. These can include various types of settings selections that are entered by an individual sonographer based on factors such as personal preference, specialty, experience level, training, system familiarity, and other factors.

Examples of some of the ultrasound parameters that are typically adapted by the sonographer to task requirements on a per-exam basis during the operator interaction sequence include dynamic range, acoustic signal gain, transmit frequency, choice of harmonic imaging versus fundamental-only imaging, time gain compensation (TGC) setting, preference for triplex versus duplex Doppler modes, and other settings.

Applicants describe two example cases to illustrate the usefulness of having customized operator profiles to support setup for different users of the sonography system.

### EXAMPLE 1

Sonographer A has significant experience with the particular sonography equipment and has demonstrated considerable skill in using Doppler scanning techniques. Thus, sonographer A is often assigned challenging cases requiring Doppler scans. Because operator setup for Doppler modes can be a time-consuming part of the interaction sequence and can necessitate a number of sequential commands and keystrokes, there would be advantages to reconfiguration of the user interface for this sonographer. A custom system configuration suitable for sonographer A can position Doppler-related controls more favorably for ease of visibility and access during operation, for example. Position, screen location, and relative size of displayed controls, menus and informational and instruction entry fields can be modified, adapted in order to enable a more efficient workflow. Hierarchical menu and sub-menu structures can be re-arranged in a custom system configuration in order to present menu and sub-menu entries of more relevance to Doppler operation so that they are more readily visible and accessible. Some exemplary Doppler-related controls that can be highlighted or rearranged for this purpose can include Doppler gain, Doppler frequency, PRF, wall filter, baseline shift, sample volume location, color box dimensions, and steering angle.

### EXAMPLE 2

Sonographer B can be more representative of operators who are less experienced and may be less specialized than sonographer A. Sonographer B can be expected to perform a range of exams in a variety of system modes. Custom system configurations suitable for more general system use that serve sonographer B can present multiple user controls for more generalized imaging modes, such as B-mode or Color Doppler imaging mode. For such a user, it can be particularly advantageous to track operator settings and instructions in the operator interaction sequence using machine learning in order to identify operator tendencies, patterns, and preferences from the interaction sequence. A custom system configuration can then be developed that re-positions operator controls at favorable locations on the user interface console in order to support workflow for sonographer B functions.

It can be appreciated that, in practice, it can be tedious to optimally establish suitable values for each operator using manual entry methods.

To provide operator customization, an embodiment of the present disclosure provides the capability to maintain and update an operator profile that stores information related to operator activity for ongoing operation of the system and maintenance of a custom system configuration. Applicants have recognized that machine learning and related pattern recognition can be applied to ultrasound system operation to improve aspects of the operator experience using a systematic approach that records operator actions from the interaction sequence and relates these to the operator identity for performing specific tasks with the system.

In conventional operation, the ultrasound operator interaction sequence begins with the operator setting up the system and initiating scanning, without system login or in any way revealing operator identity information. Ultrasound systems that do not have some type of login capabilities are not enabled to track the interaction sequence and to link user actions with any type of user profile information. Consequently, information from the interaction sequence is not available for data analytics that could be used to help improve workflow, reduce operator stress, and reduce the number of keystrokes needed in order to perform standard operations.

Applicant has recognized that, with operator login or using an identifying badge or badge reader or other device that identifies the operator and is in signal communication with the ultrasound system, the operator activity during the interaction sequence can be recorded and analyzed, with these actions consistently correlated to operator identity. Patterns for system interaction can be identified and used to help in configuring the operator interface so that it is better suited to the preferences and workflow sequences of each individual operator.

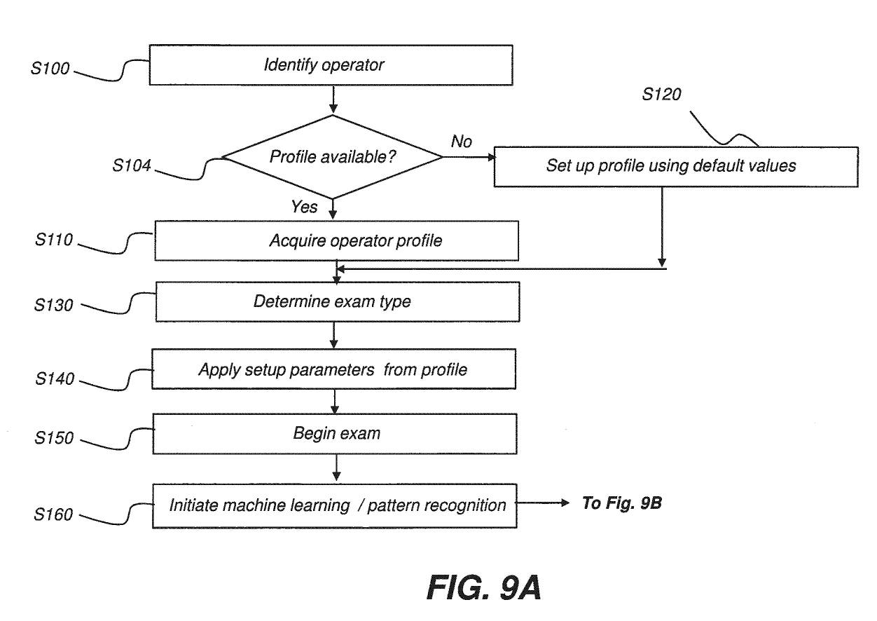

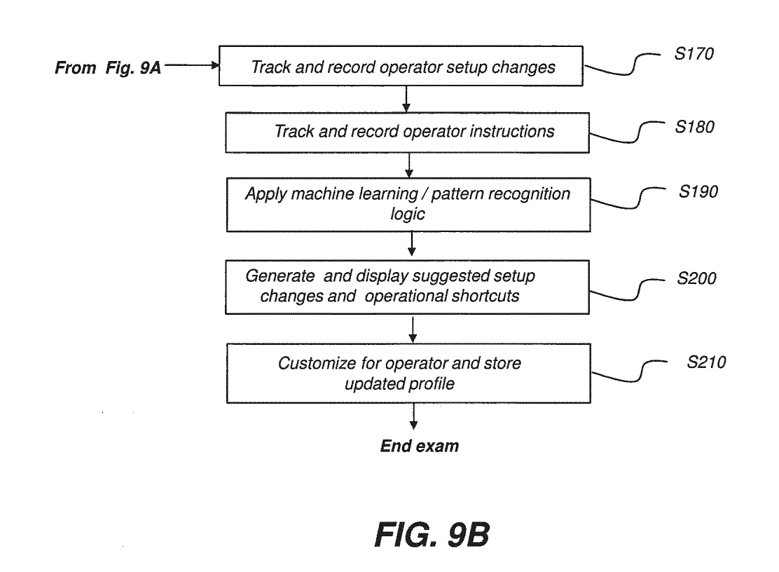

The logic flow diagrams of FIGS. 9A and 9B show a sequence for customization of ultrasound system configuration and operation using operator profile data for improved workflow.

In an identification step S100, the system identifies the operator as described previously, either through a login entry or using a badge or other scanned or sensed device. A check step S104 checks system memory to determine whether or not the identified user has an existing profile already setup and including a custom system configuration. If there is an existing profile with the appropriate data, the sequence continues to a profile acquisition step S110 in which the existing operator profile is loaded and its default settings from the custom configuration applied to the keyboard and display 14 and other elements of the user interface (UI) of the system. If there currently is no custom profile set up for the identified operator, a profile setup step S120 executes, forming a custom profile for the identified operator and applying system default values as a starting-point for system operation.

Continuing with the FIG. 9A sequence, an exam type determination step S130 determines, from operator entries or from information provided from some other source, the exam type that will be performed. A setup parameters application step S140 applies the setup parameters from the existing custom system configuration or using new profile data. A begin examination step S150 then begins the ultrasound exam with the profile settings applied. An initiate machine learning step S160 initiates the machine learning software module for monitoring and recording operator instructions during the interaction sequence on the graphical user interface (GUI) for the current exam.

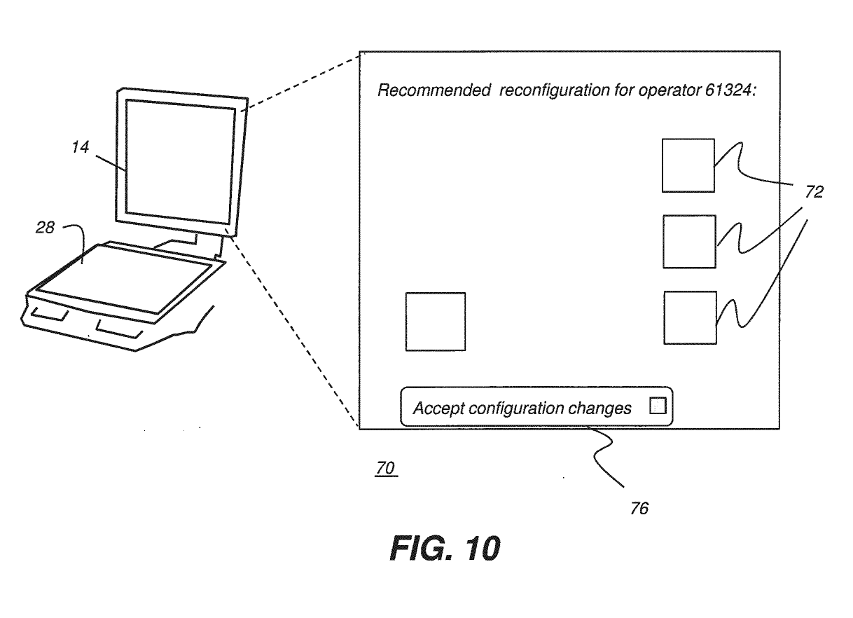

Continuing to FIG. 9B, a setup monitoring step S170 tracks and records any operator changes to ultrasound system setup during the interaction sequence. An instruction tracking step S180 begins the ongoing process of recording the interaction sequence of operator instructions that are entered during the course of the exam. A machine learning step S190 applies machine learning and pattern recognition logic to the recorded interaction sequence. At the conclusion of the exam, a generate changes step S200 can display text and graphical content to the operator for interface modifications based on the interaction sequence of operator setup and operation instructions. The generated changes that are suggested can relate to system setup commands or to the sequence of operator instructions. These results from the operator can then be compared with previous or other existing interaction sequences for the purpose of determining what types of changes would be useful for improving operator workflow for the particular type of exam noted. Generate changes step S200 can show proposed changes to the equipment setup using display screen 14 as shown schematically in FIG. 10. In the example shown herein, a display screen 70 shows a number of control buttons 72 often used by the operator, singly or in a defined interaction sequence, are displayed. The order, size, or other arrangement aspect of control buttons 72 can vary widely, depending on what best supports operator workflow. Where a known sequence of control button presses is typically used, the operator interface can highlight the next or most likely control button in sequence for standard operation. In a customization step S210, the operator is given an option to accept or to not use the projected custom system configuration changes, such as by pressing a control button 76.

According to an embodiment of the present disclosure and as shown in FIG. 10, display 14 and, optionally, a control console 28 can provide both touch screen command entry devices. Repositioning of icons, control buttons, and supporting text can be performed based on operator actions for any number of previously executed exams.

As shown in FIGS. 9A through 10, Applicants' ultrasound system and method can include a machine learning/pattern recognition module that can continually observe, track, and record operator interaction with the system in order to detect workflow patterns used by a particular operator. Advantageously, the obtained data is correlated with the operator identity.

Based on the patterns it observes in the user's actions (e.g. sequence of key strokes, equipment settings, and feature usage) during the interaction sequence the method described with reference to FIGS. 9A and 9B can propose new individualized layout(s) and can offer one-touch operation as part of the system configuration sequence for frequently used commands.

In particular, an embodiment of the disclosed system and method tracks frequency and sequencing of operator interface functions in the interaction sequence to perform various setup and imaging tasks. The system logic stores data relevant to operator activity and makes recommendations to the operator for suitable optimization of the user interface. Recommendations can include, for example, changes to the layout of a “soft” displayed keyboard or keypad; modified placement of control buttons, icons, and display windows; keyboard shortcuts; sizing, arrangement, or presentation of control panels, and other features of the operator interface.

Embodiments of the present disclosure can be facilitated by employing a software module that resides on the ultrasound system, and runs in the background, such as continuously during setup and imaging operation. The software module can track the operator interaction sequence, determine how well an existing operator interface serves the operator, and identify changes that can be advantageous. Recommendations to the operator can be provided periodically, such as during or following an imaging session, for example.

In the following description, a preferred embodiment of the present invention is described as a software program. Those skilled in the art will recognize that the equivalent of such software may also be constructed in hardware. Because image manipulation algorithms and systems are well known, the present description will be directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

An embodiment of the present disclosure can be implemented as a software program. Those skilled in the art will recognize that the equivalent of such software may also be constructed in hardware. Because image manipulation algorithms and systems are well known, the present description will be directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

A computer program product may include one or more storage media, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The methods described above may be described with reference to a flowchart. Describing the methods by reference to a flowchart enables one skilled in the art to develop such programs, firmware, or hardware, including such instructions to carry out the methods on suitable computers, executing the instructions from computer-readable media. Similarly, the methods performed by the service computer programs, firmware, or hardware are also composed of computer-executable instructions.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim.

In the following claims, the terms “first,” “second,” and “third,” and the like, are used merely as labels, and are not intended to impose numerical requirements on their objects.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

1. A method, comprising:
tracking a user's operation of an ultrasound system;
based on the tracked operations, using a machine-learning module to generate a proposed custom system configuration for the ultrasound system for the user;
presenting the proposed custom system configuration to the user; and
in response to a user instruction, implementing the proposed custom system configuration on the ultrasound system.

2. The method of claim 1 wherein tracking comprises recording and analyzing a sequence in which one or more operator interface functions are entered by the user.

3. The method of claim 1 wherein presenting the proposed configuration comprises displaying a touch screen layout or modified interaction sequence.

4. The method of claim 1 further comprising identifying the user according to a login procedure.

5. The method of claim 1 wherein tracking the user's operation comprises recording system settings.

6. The method of claim 1 wherein tracking the user's operation comprises recording a progression of operating modes used for an exam.

7. A method comprising:
identifying an operator of an ultrasound imaging apparatus according to a login procedure;
acquiring an operator profile for the identified operator;
determining a type of ultrasound exam to be performed by the operator;
applying setup parameters for the exam according to the operator profile;
tracking and recording operator instruction entry during the ultrasound exam;
applying machine learning to generate and display one or more recommended changes to the operator profile or to equipment settings according to the tracking and recording operation; and
responding to an operator instruction to accept the recommended changes and to store the changes in the operator profile.

8. The method of claim 7 wherein the operator profile includes an authorization level that controls transportation of the ultrasound imaging apparatus.

9. A method comprising:
recording an interaction sequence for settings and instructions entered by an operator in using an ultrasound system;
generating and storing a custom system configuration for the operator according to the recorded interaction sequence; and
implementing the custom system configuration when the operator uses the ultrasound system.

10. A computer storage medium having instructions stored therein for causing a computer to perform the method of claim 1.
